(19) United States
(12) Patent Application Publication
(10) Pub. No.: US 2020/0405023 A1
(43) Pub. Date: Dec. 31, 2020

(54) TSK - First Class

(71) Applicant: Peter Thomas Cook, Winter Garden, FL (US)

(72) Inventor: Peter Thomas Cook, Winter Garden, FL (US)

(73) Assignee: Refreshed Traveler, Orlando, FL (US)

(21) Appl. No.: 15/931,044

(22) Filed: May 13, 2020

Related U.S. Application Data
(63) Continuation-in-part of application No. 16/869,409, filed on May 7, 2020.
(60) Provisional application No. 62/921,594, filed on Jun. 27, 2019.
(60) Provisional application No. 62/922,255, filed on Jul. 31, 2019.

(51) Int. Cl. A45C 3/00; A45C 11/00; A61K 36/906; A61K 36/886; A62B 23/02; A61F 9/04; A61F 11/08; A61L 2/00; A61L 2/22; A47G 9/02; A47G 21/18

(52) U.S. Cl. CPC A45C 3/001; A45C 11/00; A61K 36/906; A61K 36/886; A62B 23/025; B08B 1/006; A61F 11/08; A61L 2/0088; A61L 2/22; A47G 9/0253; A47G 21/18; A61F 9/045

(57) ABSTRACT

A travel kit including a travel bag comprising a sealing device configured to seal the top of the travel bag, sealing the contents of the travel bag, the contents comprising five or more travel products.

This application is a continuation-in-part of U.S. patent application Ser. No. 16/869,409 filed on May 7, 2020, which claims priority to U.S. Provisional Patent Application No. 62/921,594 filed on Jun. 27, 2019. This application also claims priority to U.S. Provisional Patent Application No. 62/922,255 filed on Jul. 31, 2019.

### FIELD OF THE INVENTION

The present disclosure relates to a kit and more particularly to an application specific kit and its related method.

### BACKGROUND OF THE INVENTION

The present invention relates to a portable beauty, health, safety, and First Class kit. More specifically, a travel bag constructed of leather, suede, polyvinyl chloride (PVC), vinyl, rubber, fabric or cloth, polypropylene, nylon, rayon or spandex like material that has a button, buckle, zipper, zip lock, Velcro snaps, tie or locking channel that seals the top of the container/bag/kit sealing the 5 or more travel essential products inside.

Living in an intimately and overpopulated world the need for a travel kit that has all the essentials you need has never been greater. The explosion in travel has created opportunities like never seen before in the world. With cheap flight, we live in an interconnected world where with at the click of a button you can have a cheap flight across the globe. The opportunity to travel and explore has never been greater! With discount and budget airlines, cruise ships, hotel and now a gig economy with riding sharing and Airbnb traveling has never been easier with more opportunities. People travel more often now than ever before. The need to stay relaxed, healthy/beautiful, comfortable and first class on the go. People are constantly on the go and the world is overcrowding and a travel kit with the 5 or more essentials you need in a convenient grab and go TSK—First Class travel kit.

Conventional kits and first aid kit are marketed and sold for wound care and emergencies. They typically include bandages and medical supplies. These components are typically generic and designed to address a wide range of injuries and conditions and conventional kits have a wide arrange of applications. Because conventional kits are generic and may be used in virtually any household or business, with these kits often include bandages, medications, and/or a range of products. Likewise, such kits often contain medication.

Emergency and germs kits are specific and limited in scope, these are typically housed in containers made of polyethylene and have a wide range in size and color. Products are typically single use and specific in use, wounds, flu, germs, medical care. Likewise, such kits often include a range of products designed for medical conditions and/or to kill flu virus.

The present invention provides a single kit, the TSK—First Class which has the 5 or more essential travel products inside a travel bag that seals. It is invented to help one relax, stay healthy/beautiful, comfortable and have a First Class traveling experience. Due to the overall size and restrictions and limitations of other kits, we have invented the TSK—First Class and the summary are intended for purposes of illustration only and are not intended to limit the scope of the present disclosure.

### SUMMARY OF THE INVENTION

TSK—First Class is an application specific for the relaxation, healthy/beauty, comfort and a First Class travel experience. The TSK—First Class with its five essential travel products in a Travel Bag constructed of the following materials leather, suede, polyvinyl chloride (PVC), vinyl, rubber, fabric or cloth, polypropylene, nylon, rayon or spandex with a button, buckle, zipper, zip lock, Velcro, snaps, tie or locking channel that seal the top of the container/travel bag. This Grab & Go filled with reusable and multi-pack travel essential items is necessary for health, wellness, comfort, and beauty when traveling. The essential products include: face sheet mask (24K gold, hydration-collagen, K-Beauty), multi-pack biodegradable wet wipes, multi-pack facial tissues, silk or satin pillowcase, facial cleaning towelettes, hydration mist, eye mask, lip & eye covers, socks/booties/foot covers, hand sanitizer, face mask; N95 or KN95 respirator, lip balm, hand cream, earphones and/or ear buds, toilet seat sanitizer, straws, essentials oils, ginger drops, ear plugs, breath spray and/or breath mints, sunscreen, aloe and/or spray disinfectant to keep a traveler's relax, stay healthy/beautiful, comfortable, and travel First Class.

Further, below you will find specific examples intended for the purposes of illustration and are not intended to limit the scope of the present disclosure. Other items can and will be added as they are invented and become available.

### BRIEF DESCRIPTION OF THE DRAWINGS

The drawings described herein are for the illustrative purposes only and may be selective embodiments and operation of the invention, and not all possible implementations, and are not intended to limit the scope of the present disclosure.

FIG. 1 is a front elevation view of a travel bag/container according to an embodiment of the invention.

FIG. 2 is a front elevation view of a travel size hand cream that may be comprised by a travel bag according to an embodiment of the invention.

FIG. 3 is a front elevation view of a package of moist toilettes/wet wipes that may be comprised by a travel bag according to an embodiment of the invention.

FIG. 4 is a front elevation view of a travel size package of tissues that may be comprised by a travel bag according to an embodiment of the invention.

FIG. 5 is a front elevation view of a travel pillow case that may be comprised by a travel bag according to an embodiment of the invention.

FIG. 6 is a front elevation view of a travel size package of moisturizing wipes that may be comprised by a travel bag according to an embodiment of the invention.

FIG. 7 is a front elevation view of a hydrating mist dispenser that may be comprised by a travel bag according to an embodiment of the invention.

FIG. 8 is a front elevation view of an eye mask that may be comprised by a travel bag according to an embodiment of the invention.

FIG. 9 is a front elevation view of foot covers that may be comprised by a travel bag according to an embodiment of the invention.

FIG. 10 is a front elevation view of head phone/ear phone devices that may be comprised by a travel bag according to an embodiment of the invention.

FIG. 11 is a front elevation view of a travel-sized container of hand sanitizer that may be comprised by a travel bag according to an embodiment of the invention.

FIG. 12 is a front elevation view of a face mask that may be comprised by a travel bag according to an embodiment of the invention.

FIG. 13 is a front elevation view of an N95/KN95 respirator that may be comprised by a travel bag according to an embodiment of the invention.

FIG. 14 is a front elevation view of a packaged lip balm that may be comprised by a travel bag according to an embodiment of the invention.

FIG. 15 is a front elevation view of a travel sized spray disinfectant dispenser that may be comprised by a travel bag according to an embodiment of the invention.

FIG. 16 is a front elevation view of a toilet seat sanitizing dispenser that may be comprised by a travel bag according to an embodiment of the invention.

FIG. 17 is a front elevation view of a sheet mask of one type of 24K gold, hydration-collagen, and K-Beauty mask that may be comprised by a travel bag according to an embodiment of the invention.

FIG. 18 is a front elevation view of a package of sunscreen that may be comprised by a travel bag according to an embodiment of the invention.

FIG. 19 is a front elevation view of an aloe dispenser that may be comprised by a travel bag according to an embodiment of the invention.

FIG. 20 is a front elevation view of a container of ginger drops that may be comprised by a travel bag according to an embodiment of the invention.

FIG. 21 is a front elevation view of a breath spray dispenser and a breath mint container, as well as breath mints, either or both of which may be comprised by a travel bag according to an embodiment of the invention.

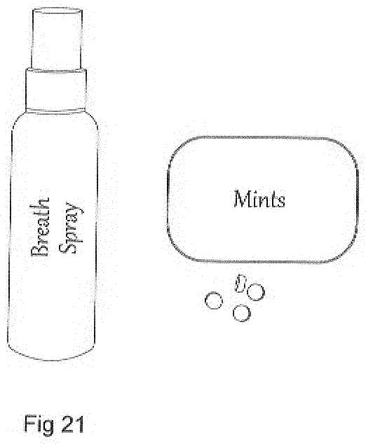

FIG. 22 is a front elevation view of an essential oils dispenser that may be comprised by a travel bag according to an embodiment of the invention.

FIG. 23 is a front elevation view of a package of ear plugs and two ear plugs that may be comprised by a travel bag according to an embodiment of the invention.

FIG. 24 is a front elevation view of a straw that may be formed from plastic, metal, wood, bamboo, or paper that may be comprised by a travel bag according to an embodiment of the invention.

FIG. 25 is a front elevation view of an inventory list of the contents of a travel bag comprised by a travel bag according to an embodiment of the invention.

FIG. 26 is a front elevation view of a travel bag consisting of hand cream, a package of wet wipes, a travel pillow case, a package of moisturizing wipes, and a package of tissues according to an embodiment of the invention.

FIG. 27 is a front elevation view of a travel bag consisting of a hydration mist dispenser, socks/foot covers, hand sanitizer, earphones and an eye mask according to an embodiment of the invention.

FIG. 28 is a front elevation view of a travel bag consisting of lip balm, a face mask, an N95/KN95 respirator, spray disinfectant, and toilet seat sanitizer according to an embodiment of the invention.

FIG. 29. is a front elevation view of a travel bag consisting of sunscreen, aloe, a collagen mask, breath spray/mints, and ginger drops according to an embodiment of the invention.

FIG. 30 is a front elevation view of a travel bad consisting of essential oils, straws, an eye mask, ear plugs, and lip balm according to an embodiment of the invention.

FIG. 31 is a front elevation view of an inventory list of the contents of a travel bag comprised by a travel bag according to another embodiment of the invention.

FIG. 32 is a front elevation view of a diagram showing the 24 items that may be comprised by a travel bag according to an embodiment of the invention, the travel bag being constructed of at least one of leather, suede, polyvinyl chloride (PVC), vinyl, rubber, fabric or cloth, polypropylene, nylon, rayon or spandex like material and comprising a button, buckle, zipper, zip lock, Velcro, snaps, tie or locking channel that seal a top portion of the container thereby sealing the contents inside.

### DETAILED DESCRIPTION OF THE INVENTION

The invention and accompanying embodiments will be described in detail the specific embodiments more fully with reference to the drawing illustrated and described.

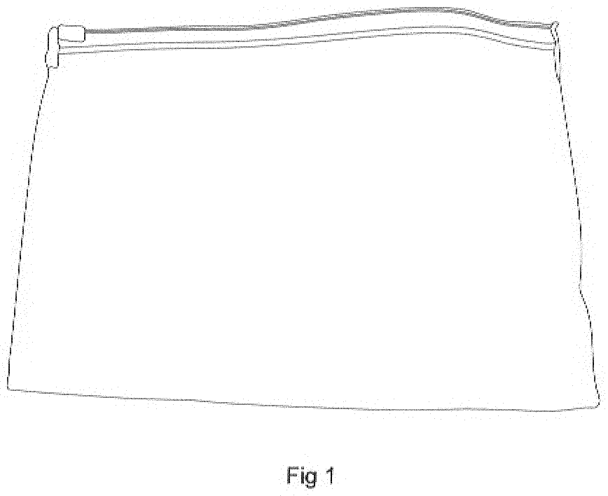

With reference to the figures, a kit 100 as shown in FIG. 1 is a diagramed drawing and shows the embodiments of this travel bag constructed of the following materials leather, suede, polyvinyl chloride (PVC), vinyl, rubber, fabric or cloth, polypropylene, nylon, rayon or spandex with a button, buckle, Zipper, zip lock, Velcro, snaps, tie or locking channel that seal the top of the container/travel bag.

The remaining components of the kit are shown outside for illustration. These 24 travel essentials include the following components:

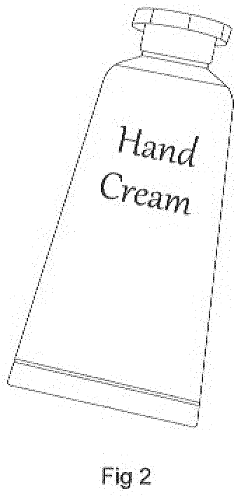

FIG. 2 depicts a travel-sized 3 oz. container 200 of hand cream that may be comprised by a kit according to an embodiment of the invention.

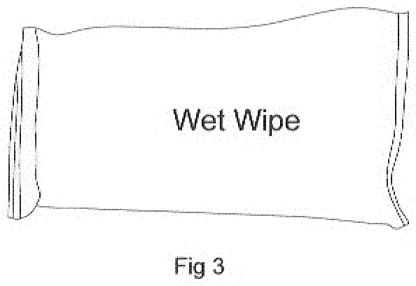

FIG. 3 depicts a package of wet wipes 300 that may be comprised by a kit according to an embodiment of the invention.

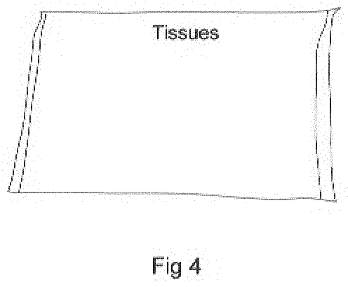

FIG. 4 depicts a packaged 8 to 10 count soft white 3 or 4-ply facial tissues 400 that may be comprised by a kit according to an embodiment of the invention.

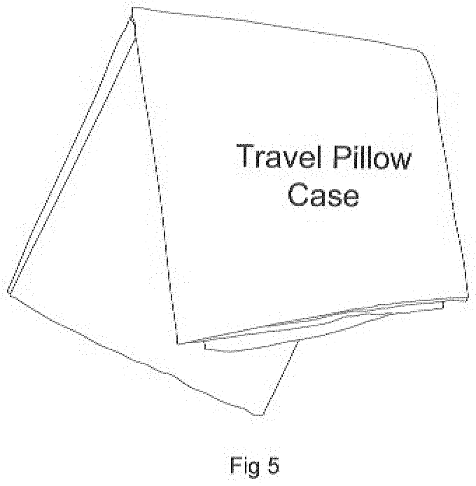

FIG. 5 depicts a travel size pillowcase 500 made of silk or satin that may be comprised by a kit according to an embodiment of the invention. The pillowcase 500 can be filled with a travel pillow or with clothing during a trip for comfort. It can also double as a headrest as well as a face mask.

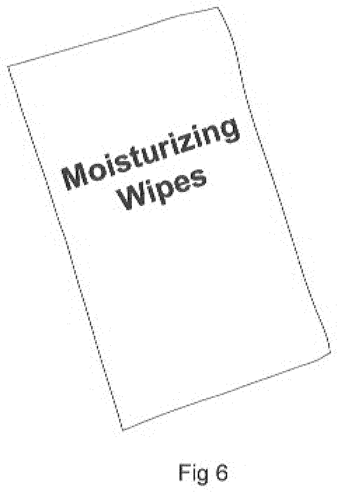

FIG. 6 depicts a packet of moisturizing wipes 600 that may be comprised by a kit according to an embodiment of the invention. These disposable wipes 600 are infused with moisturizing body lotion to revive, regenerate, and repair the skin.

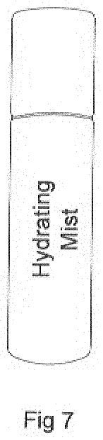

FIG. 7 depicts a travel hydrating mist 700 that may be comprised by a kit according to an embodiment of the invention. The hydrating mist 700 may be a refreshing spray, purifying mist in a size of no less than 1 oz and no more than 3 oz. to prevent a dry skin and help maintain a healthy complexion when traveling.

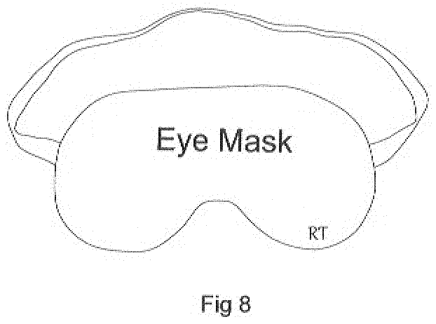

FIG. 8 depicts a travel eye mask 800 that may be comprised by a kit according to an embodiment of the invention. The travel eye mask 800 may be individually packaged, made of satin or silk and designed to be light for rest, sleep and relaxation when traveling. The mask 800 will have an adjustable head band for comfort.

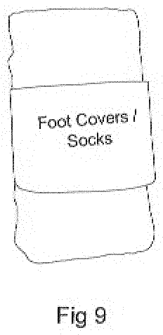

FIG. 9 depicts foot covers 900, commonly known as booties or socks, that may be comprised by a kit according to an embodiment of the invention. These covers 900 of your feet or shoes can be reusable or disposable. Single or multiple use, for travelers to protect their feet when walking through TSA security, or taking their shoes off on a plane, elevation has been known to cause swelling in your feet.

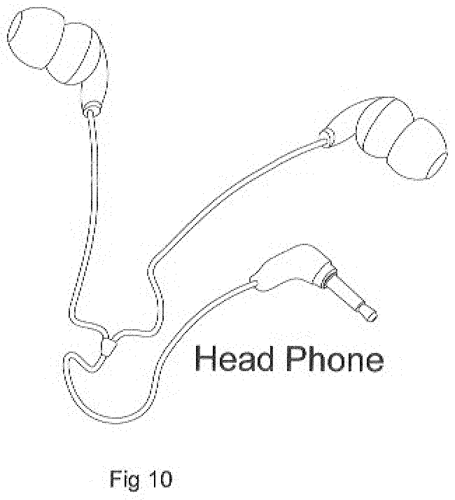

FIG. 10 depicts earphones and/or ear buds 1000 that may be comprised by a kit according to an embodiment of the invention. The ear buds 1000 may be wireless or hardwired for listening to those essential airline announcements or plugging into the in-flight entertainment system.

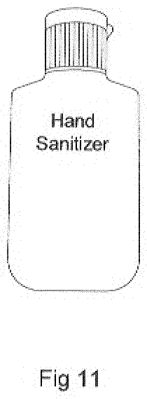

FIG. 11 depicts a 2 oz bottle of antibacterial hand sanitizer with aloe 1100 that may be comprised by a kit according to an embodiment of the invention. With airlines known to infrequently dean their plane, having a waterless hand cleaning option is always helpful for healthy/beautiful and comfortable on the go. The bottle containing the sanitizer will be made from polyethylene terephthalate (PET) and preferably of recycled material.

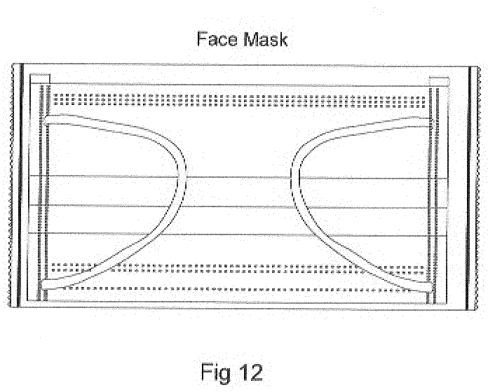

FIG. 12 depicts a fashion face mask 1200 or 3, 4 or 5-ply face mask that may be comprised by a kit according to an embodiment of the invention. The mask 1200 may keep from breathing the recycled air on airplanes and to stay healthy/beautiful and comfortable on your travels. Some airlines now mandate face masks.

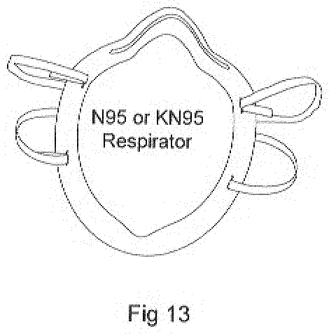

FIG. 13 depicts an N95/KN95 respirator 1300 that may be comprised by a kit according to an embodiment of the invention. The respirator 1300 may be used to keep from breathing the recycled air on airplanes and to stay healthy/beautiful and comfortable on your travels.

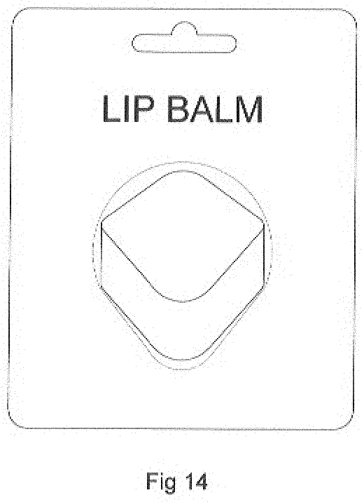

FIG. 14 depicts a package or tube of lip balm 1400 that may be comprised by a kit according to an embodiment of the invention. The lip balm 1400 may be used to nourish and moisturize your lips. The lip balm 1400 may be packaged or in a tube that self-packaged. The lip balm 1400 is for convenience when traveling to keep your lips from cracking in the dry air or airplane or in different climates.

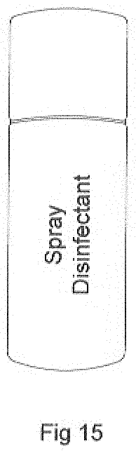

FIG. 15 depicts a travel size spray disinfectant 1500 that is no more than 3 oz. in size and made with both natural and/or chemicals and designed to inactivate or destroy microorganisms on inert surfaces.

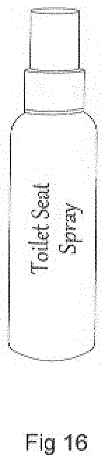

FIG. 16 depicts a toilet seat spray 1600 that may be comprised by a kit according to an embodiment of the invention. Traveling does not leave you much room for choosing when nature calls and toilet seat sanitizer 1600 offers you the protection and safety you need to have a first class bathroom experience.

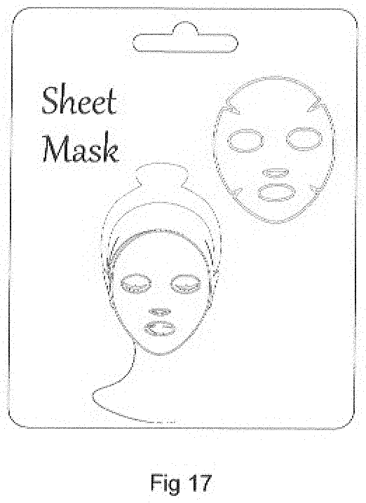

FIG. 17 depicts a sheet mask or collagen mask 1700 that may be comprised by a kit according to an embodiment of the invention. The masks 1700 can be face and beauty masks, 24K gold face masks, K-Beauty face masks, hydration masks and collagen masks. These masks 1700 prevent lines, reduce redness, and eradicate dryness ai! common effects from travel especially air travel.

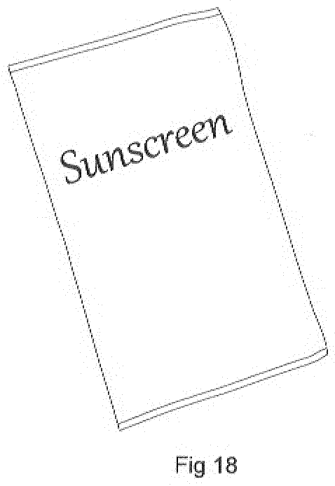

FIG. 18 depicts a sunscreen that may be comprised by a kit according to an embodiment of the invention. These can be a single packet, sunscreen pen, travel size or full-size sunscreen product. Traveling is often to sunny destination and with the ozone layer slowing fading the need for sunscreen is greater than ever. Skin cancer kills 2 people in the US every hour and 1 in 5 Americans will develop skin cancer before the age of 70.

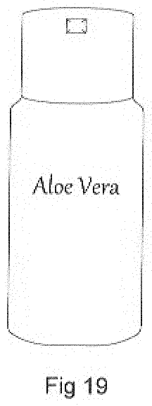

FIG. 19 depicts a container of aloe vera and/or aloe-based product 1900 that may be comprised by a kit according to an embodiment of the invention. The aloe-based product 1900 may be used to treat sunburns and other skin wounds. This could be a single packet or a larger container inside the travel bag.

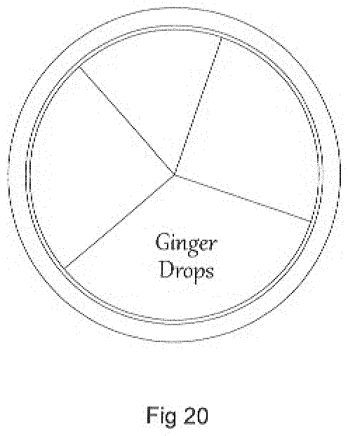

FIG. 20 depicts a package of ginger drops 2000 that may be comprised by a kit according to an embodiment of the invention. Ginger is a natural product know to reduce nausea or motion sickness. This can be in a power, capsule, drop, liquid or other form of ginger to naturally reduce the effects of motion sickness when traveling.

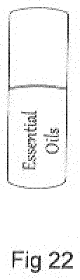

FIG. 22 depicts a bottle of an essential oil 2200 that may be comprised by a kit according to an embodiment of the invention. There are 22 different essential oils in the kit. Essential oils have been known to provide respiratory disinfection, decongestant, and psychological benefits. They include lavender, ginger, peppermint, tea tree, lemongrass, eucalyptus, bergamot, frankincense. patchouli, cinnamon, chamomile, clary sage, cedar wood, oregano, orange rosemary, geranium, cypress, copaiba, lemon, or dove.

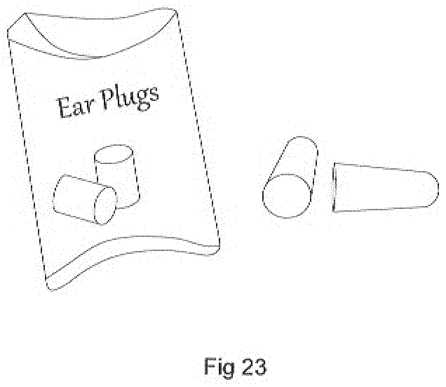

FIG. 23 depicts ear plugs 2300 that may be comprised by a kit according to an embodiment of the invention. With screaming kids and crying babies, ear plugs 2300 are a travel must. To escape the noise and relax, rest and get sleep these sound reducing ear plugs 2300 are an essential for a traveler.

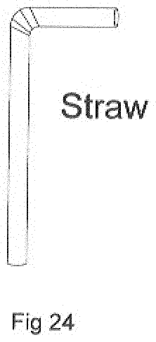

FIG. 24 depicts a straw 2400 that may be comprised by a kit according to an embodiment of the invention. Made from plastic, bamboo, wood, metal or paper, the straw 2400 can be reusable or one-time use.

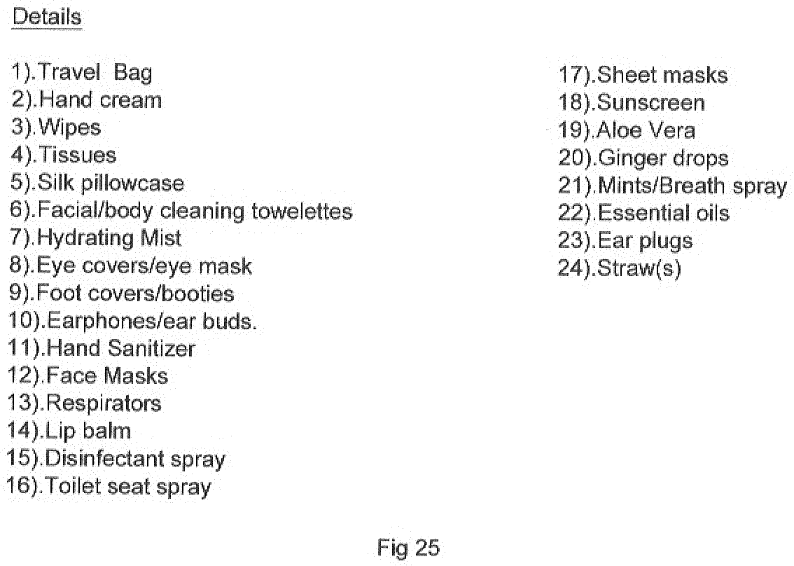

FIG. 25 depicts a detailed list 2500 of all potential items that may be comprised by a kit according to an embodiment of the invention.

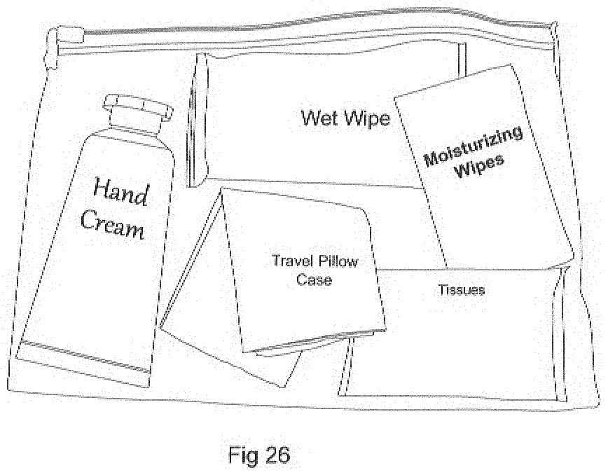

FIG. 26 depicts a kit 2600 according to an embodiment of the invention. In the present embodiment of the travel kit 2600, a bag 2602 comprises a travel size hand cream (3 oz or less) 2604, a multi-pack of wet wipes 2606, a multi-pack of facial tissues 2608, a package of moisturizing wipes 2610, and a satin/silk travel pillowcase 2612.

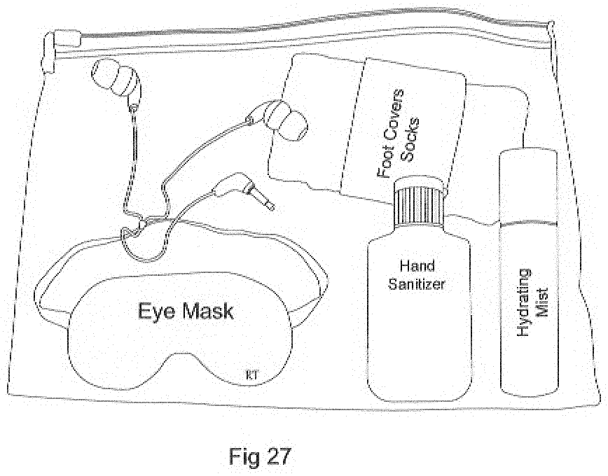

FIG. 27 depicts a kit 2700 according to an embodiment of the invention. The present embodiment comprises a bag 2702 containing an eye mask 2704, ear plugs 2706, a travel size hand sanitizer 2708, a hydration mist 2710, and a pair of socks/foot covers 2712.

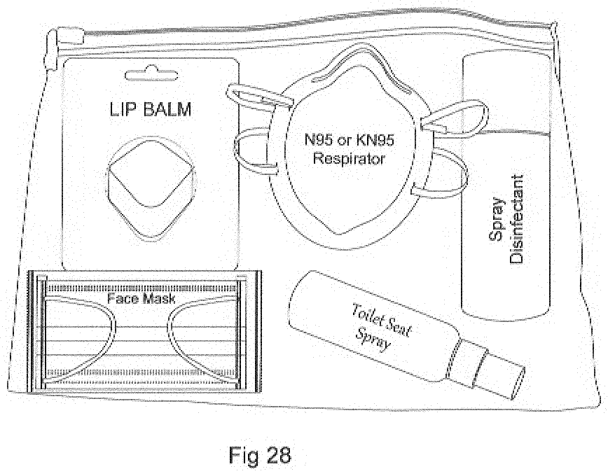

FIG. 28 depicts a kit 2800 according to another embodiment of the invention. The present embodiment comprises a bag 2802 containing lip balm 2804, KN95 and/or N95 respirator 2806, 3, 4, or 5-ply or fashion face mask 2808, toilet seat sanitizer 2810, and spray disinfectant 2812 inside the travel bag 2802 constructed of the leather, suede, polyvinyl chloride (PVC), vinyl, rubber, fabric or cloth, polypropylene, nylon, rayon or spandex with a button, buckle, zipper, zip lock, Velcro, snaps, tie or locking channel 2814 that seals the top keeping the contents safely and securely inside.

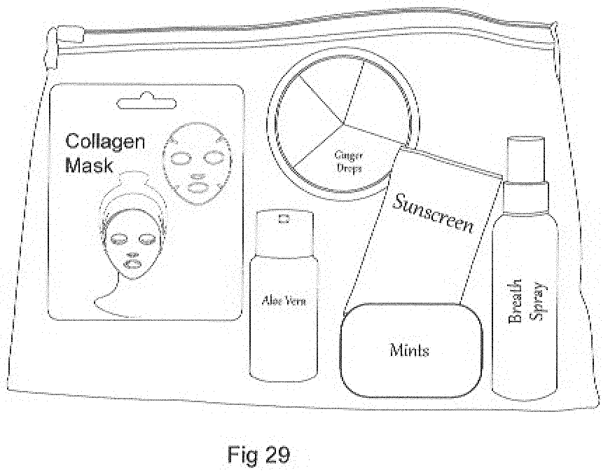

FIG. 29 depicts a kit 2900 according to another embodiment of the invention. The present embodiment comprises a bag 2902 containing sheet mask/collagen masks 2904, a container of aloe vera 2906, a container of ginger drops 2908, a package of sunscreen 2910, a container of mints 2912, and a breath spray dispenser 2914, the travel bag 2902 being constructed of the leather, suede, polyvinyl chloride (PVC), vinyl, rubber, fabric or cloth, polypropylene, nylon, rayon or spandex with a button, buckle, zipper, zip lock, Velcro, snaps, tie or locking channel 2916 that seal the top of the container/travel bag.

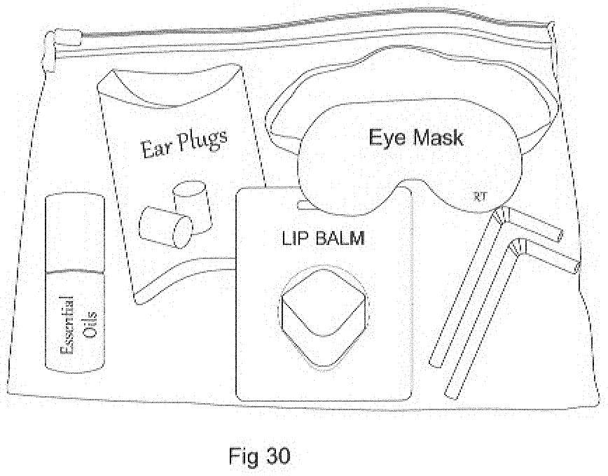

FIG. 30 depicts a kit 3000 according to another embodiment of the invention. The present embodiment comprises a bag 3002 containing essential oils 3004, ear plugs 3006, lip balm 3008, eye mask 3010, and a plurality of straws 3012.

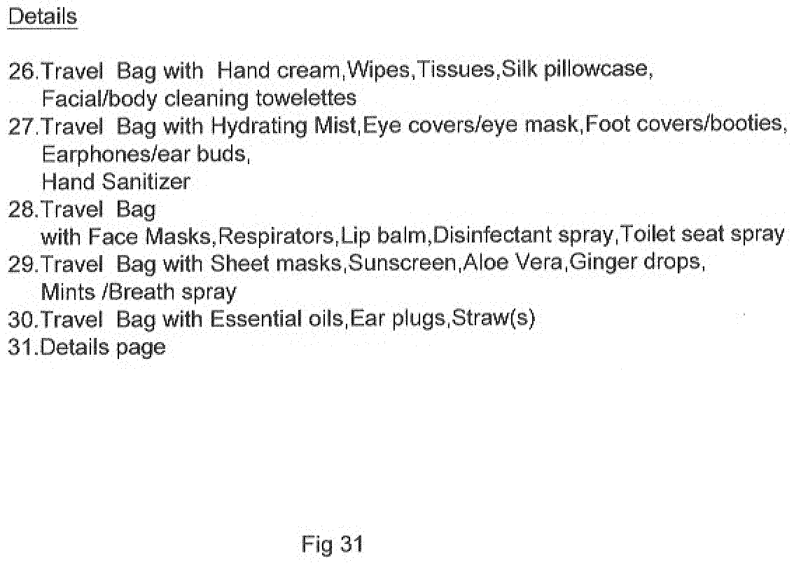

FIG. 31 depicts a detailed list 3100 of an exemplary list of the items that may be comprised by a kit according to an embodiment of the invention. These depictions detail out several ways that the kits according to the inventions can be configured and used. The combination of products inside the kit will vary based on individual user preference and need as shown in the detailed depictions.

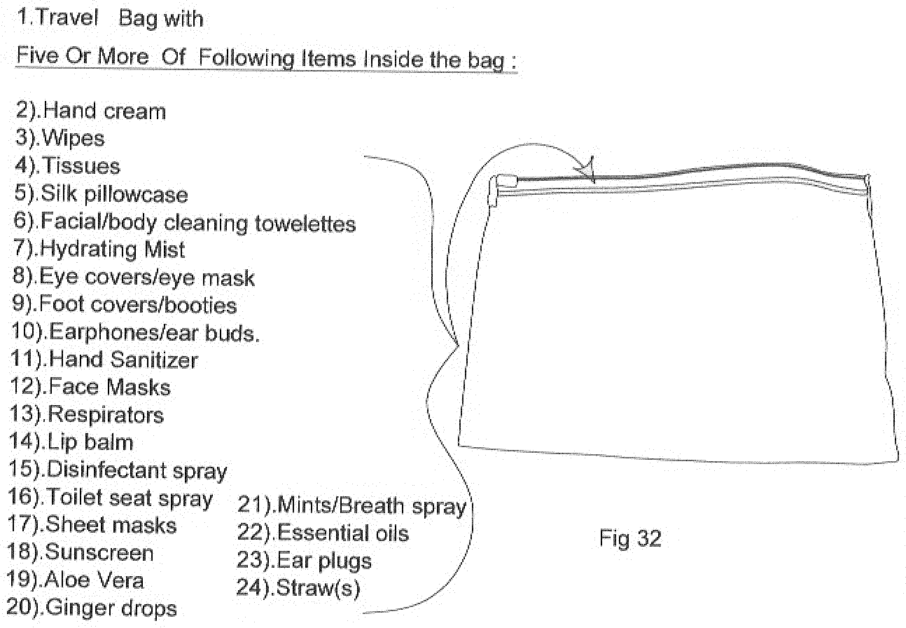

FIG. 32 depicts assembly instructions 3200 that may be comprised by a kit according to an embodiment of the invention.

What is claimed is:

1. A travel kit comprising: a travel bag comprising a sealing device configured to seal the top of the travel bag, sealing the contents of the travel bag, the contents comprising five or more travel products.

2. The kit of claim 1, wherein said travel bag is fabricated from at least one of leather, suede, polyvinyl chloride (PVC), vinyl, rubber, fabric, cloth, polypropylene, nylon, rayon, or spandex.

3. The kit of claim 1, wherein said sealing device is at least one of a button, buckle, zipper, zip lock, Velcro, snaps, tie, or locking channel.

4. (canceled)

5. The kit of claim 1, wherein said travel bag contains at least five travel products selected from the group consisting of a travel 3 oz. or smaller container of hand cream, a package of wet wipes, a package of facial tissue, a pillowcase, facial/body cleaning towelettes, a 3 oz. or smaller container of hydrating mist, an eye mask, socks/foot cover, earphones, a 3 oz. or less container of hand sanitizer, a face mask, a KN95/N95 respirator, lip balm, a 3 oz. or smaller container of disinfectant spray, a 3 oz. or smaller container of toilet seat sanitizer, sheet/collagen mask, sunscreen, aloe, ginger, breath spray/mints, essential oils, ear plugs, and straw(s).

6. The kit of claim 1, wherein said travel bag may have a logo displayed on an outer surface thereof.

7. (canceled)
